(19) United States
(12) Patent Application Publication
(10) Pub. No.: US 2008/0026391 A1
(43) Pub. Date: Jan. 31, 2008

(54) BLOOD DIAGNOSIS METHOD FOR DIALYSIS PATIENT AND DIALYSIS MACHINE

(75) Inventors: Eiichiro Ichiishi, Sendai-shi (JP); Makoto Ishizaki, Sendai-shi (JP); Kazuhisa Fukushima, Tokyo (JP); Tsuneji Sawai, Tokyo (JP); Hidetoshi Aoki, Tokyo (JP); Atsushi Ito, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: Yokogawa Electric Corporation, Musashino-shi (JP); Tohoku University, Sendai-shi (JP)

(21) Appl. No.: 11/780,664

(22) Filed: Jul. 20, 2007

(30) Foreign Application Priority Data
Jul. 26, 2006 (JP) P2006-202680

(51) Int. Cl. C12Q 1/68; A61M 1/16

(52) U.S. Cl. 435/6; 604/29

(57) ABSTRACT

Provided is a blood diagnosis method of providing a diagnostic marker which is general and which contributes to dialysis treatment and evaluation of clinical effects. The method includes a step of collecting blood samples from a dialysis patient before and/or after dialysis and a step of performing a gene diagnosis based on mRNA markers on the collected blood samples. The mRNA markers are previously identified on the basis of correlations between clinical data and mRNA profiles.

### BACKGROUND OF THE INVENTION

1. Field of the Invention

The present invention relates to a method of making a diagnosis and performing an examination using blood collected from a dialysis patient, and a dialysis machine suitable for the method.

2. Description of the Related Art

In dialysis using a dialysis machine (for example, see Patent Literature 1), it is necessary to select dialysis membrane suitable for a patient condition, to determine the patient's primary disease, and to identify clinical progress by observing the prognosis after dialysis and monitoring complication risks such as infectious disease and malnutrition. Since dialysis patients have remarkably poor functions in terms of excreting and removing body waste and an artificial bias due to the dialysis exists after dialyzing blood, it is difficult to accurately understand the clinical results by a test using the same scale as a normal person. Accordingly, in order to understand a dialysis patient's clinical progress, for example, clinical parameters of (1) to (3) described below are used as diagnostic markers, in addition to monitoring a volume of urine, body weight, muscle mass, biochemical tests of blood, and waste removal time.

(1) Removal Rate per Hour (URR₁ₕᵣ)

The removal rate of urea nitrogen per hour is preferably 30% or more at a blood flow rate of 200 ml/min or more. When the removal rate is greater than 30%, a modification of dialysis treatment is necessary (which is based on Seventh Japanese HDF Academy).

(2) Creatinine Generation Rate (% CrGR)

A target value is 100% or more for a dialysis patient and is 90% or more for a diabetic dialysis patient. It is necessary to activate muscle metabolism and to enhance % CrGR by properly in-taking protein or exercising.

(3) Standard Dialysis Dose (Kt/V)

It is considered that the efficient removal of uremic toxins is necessary for excellent clinical performance. The prevalence rate due to the uremic toxins increases when the standard dialysis dose (Kt/V) is 0.8 or less and continuously decreases when the dose is in the range of 0.9 to 1.5. When the dose is greater than 1.5, the movement of the uremic toxins accumulated in cells or tissues to blood vessels deteriorates and the amount of uremic toxins removed from the whole body decreases. Accordingly, since the circulation dynamics is made unstable in dialysis and thus a sufficient dialysis dose is not maintained, the pathological condition of uremia is deteriorated.

[Patent Literature 1] Japanese Unexamined Patent Application, Publication No. 9-10301

[Patent Literature 2] Japanese Unexamined Patent Application, Publication No. 2005-37368

However, a complex numerical calculation based on plural clinical data is required to obtain the diagnostic markers. The operation of acquiring the clinical data or the operation of inducing the clinical markers on the basis of the clinical data is complicated and thus causes a problem in view of simplicity. The details of selection of the diagnostic markers are regarded as know-how in the respective medical centers. Generally, dialysis patients exhibit different clinical progress due to differences in primary diseases. Accordingly, it is generally difficult to select dialysis membranes suitable for the dialysis patients or to set dialysis conditions suitable for the dialysis patients. Therefore, there is a need for a diagnostic marker which is simple and general and which can be easily used on the spot, and a method of using the same. Specifically, it is necessary to replace the dialysis membranes at a proper time and it is thus difficult to select the replacement time of the membranes as well as to select the membranes. Accordingly, when a method is developed of acquiring diagnosis information as an indicator for selecting the kinds and the replacement times of the dialysis membranes, it will greatly contribute to effective dialysis treatment.

However, it has been proved that a factor indicating a patient's nutritive condition such as PEM (Protein Energy Malnutrition) is very important to control the clinical effect of a blood dialysis treatment. However, it has been reported that the factor such as PEM has a negative correlation with the conventional diagnostic markers such as a standard dialysis dose (Kt/V). Accordingly, in addition to the conventional diagnostic markers, there is a need for development of a new indicator indicating a nutritive condition. It has been reported that various inflammatory cytokines are associated with deterioration in pathological conditions of uremia of terminal patients with renal failure. Accordingly, when diagnostic markers are found which have a correlation with the generation of inflammatory cytokines and which are helpful in dialysis treatment, the dialysis treatment may be optimized or the clinical effect may be evaluated.

### SUMMARY OF THE INVENTION

An object of the invention is to provide blood diagnosis and examination methods using a diagnostic marker which is simple and general and which contributes to dialysis treatment and evaluation of clinical effects.

According to an aspect of the invention, there is provided a blood diagnosis method comprising the steps of: (1) collecting blood samples from a dialysis patient before and/or after dialysis; (2) extracting mRNAs from the collected blood samples; and (3) carrying out a gene expression profiling process on the extracted mRNAs before and/or after the dialysis.

In the blood diagnosis method, the step of (3) be performed on expression products of one or more predetermined genes by the use of a DNA microarray or a real-time PCR.

In the blood diagnosis method, the expression products of the one or more genes may include at least one selected from the group consisting of: (a) one that an expression level in a primary disease is significantly different from that of a normal person; (b) one that an expression level significantly varies depending on severities of a patient's medical condition; (c) one that an expression level significantly varies before and/or after the dialysis depending on the types of dialysis membrane used in the dialysis and (d) one that an expression level significantly varies in a prognosis.

In the blood diagnosis method, the collection of the blood samples in the step of (1) may be performed by the use of a tube properly branched from a dialyzer, which the dialysis patient's blood is made to flow in, to the outside thereof.

In the blood diagnosis method, the steps of (2) and (3) may be performed by the use of an integrated cartridge which has means for extracting the mRNAs from the blood, means for detecting the mRNAs from the blood, and individual chambers connected to each other through flow passages so as to implement the means.

According to another aspect of the invention, there is provided a blood examination method comprising the steps of: (A) extracting mRNAs from blood samples collected from a patient before and/or after dialysis; and (B) carrying out a gene expression profiling process on the extracted mRNAs before and/or after the dialysis.

In the blood examination method, the step of (B) may be performed on expression products of one or more predetermined genes by the use of a DNA microarray or a real-time PCR.

In the blood examination method, the expression products of the one or more genes may include at least one selected from a group consisting of: (a) one that an expression level in a primary disease is significantly different from that of a normal person; (b) one that an expression level significantly varies depending on severities of a patient's medical condition; (c) one that an expression level significantly varies before and/or after the dialysis depending on the types of dialysis membrane used in the dialysis; and (d) one that an expression level significantly varies in a prognosis.

In the blood examination method, the steps of (A) and (B) may be performed by the use of an integrated cartridge which has means for extracting the mRNAs from the blood, means for detecting the mRNAs from the blood, and individual chambers connected to each other through flow passages so as to implement the means.

The present invention also provides a dialysis machine comprising: an inflow line for allowing blood to flow from a dialysis patient; a dialyzer connected to the inflow line; an outflow line for allowing the blood to flow in the dialysis patient from the dialyzer; and a blood sample collection line branched from the inflow line through a valve.

In the dialysis machine, a gene analyzer may be connected to the blood sample collection line. In the dialysis machine, the gene analyzer may include an integrated cartridge which has means for extracting the mRNAs from the blood, means for detecting the mRNAs from the blood, and individual chambers connected to each other through flow passages so as to implement the means.

In the blood diagnosis and examination methods according to the invention, since the gene expression profiling process is performed on the blood samples collected before and/or after the dialysis, it is possible to make a diagnosis and an examination, which can enable easy understanding of the clinical progress of the dialysis or to accurately select a kind of a membrane used in the dialysis.

Since the dialysis machine according to the invention is configured to collect a blood sample from a line for allowing the blood collected from the patient to flow in the dialyzer, it is possible to make a diagnosis and an examination using a gene expression profiling process along with the dialysis with no burden on a patient.

### BRIEF DESCRIPTION OF THE DRAWINGS

FIG. 1 is a diagram illustrating a configuration of a dialysis machine.

### DETAILED DESCRIPTION OF THE INVENTION

Hereinafter, a blood diagnosis method according to one aspect of the invention will be described.

The present invention is based on the fact that profiles of a specific mRNA group in blood samples before and/or after dialysis of a dialysis patient have a correlation with quantitative and qualitative variations of conventional diagnostic markers. The inventor found out that it is possible to obtain useful correlation data between clinical conditions and gene diagnosis information by narrowing the range of the mRNA group so as to have a strong correlation with the conventional diagnosis markers. By using the mRNA group having a high correlation with the patient's primary disease or a specific clinical parameter as a diagnosis marker, it is possible to provide diagnosis and examination methods which enable information affecting the clinical progress of dialysis or the treatment methods to be acquired.

A blood diagnosis method according to an embodiment is performed in the following procedure. (1) A step of collecting blood samples from a dialysis patient is performed before and/or after dialysis. Subsequently, (2) a step of extracting mRNAs from the collected blood samples is performed. Finally, (3) a step of carrying out a gene expression profiling process on the extracted mRNAs before and/or after the dialysis is performed.

In the blood diagnosis method, the step of (3) may be performed on expression products of one or more predetermined genes by the use of a DNA microarray or a real-time PCR. The expression products of the one or more genes may include at least one selected from a group consisting of: (a) one that an expression level in a primary disease is significantly different from that of a normal person; (b) one that an expression level significantly varies depending on severities of a patient's medical condition; (c) one that an expression level significantly varies before and/or after the dialysis depending on the types of dialysis membrane used in the dialysis; and (d) one that an expression level significantly varies in a prognosis.

In this way, in the blood diagnosis method, a specific mRNA group in blood is used as a marker (a gene expression product to be diagnosed). The gene (expression product) group used as the marker can be selected from a variety of view points. For example, in blood samples of a chronic hepatitis patient group, a gene group which is up-regulated by the dialysis and a gene group which is down-regulated by the dialysis are previously identified as “chronic hepatitis biomarkers” and can be used in the present invention. By profiling the blood samples collected from the patient before and/or after the dialysis using probes based on arrangements of the gene groups as probes of a DNA microarray or a real-time PCR, the markers can be effectively used for the diagnosis. In this case, a probe of one or more gene arrangements not representing a variation in behavior due to the dialysis can be used as a control probe.

Similarly, by previously identifying specific gene groups of a group of diabetic patients with renal disease or a group of patients with renal disease as “a diabetic renal disease biomarker” or a “renal disease biomarker”, the markers can be used in the diagnosis method according to the present invention. By profiling the markers, it is possible to estimate the severity of the medical condition or the primary disease.

When a gene group having a high correlation with existing clinical parameters is previously selected and the expression of the gene group is profiled in the diagnosis method according to the present invention, it is possible to make a diagnosis using the gene expression profiling process without using the clinical parameters. In addition, by performing a gene expression profiling process on the gene group correlated with the creatinine generation rate, which is a clinical parameter useful as a determination indicator of a treatment effect or a patient's nutritive condition, it is possible to determine the patient's nutritive condition. Similarly, a gene group correlated with another indicator used as a conventional clinical parameter can be used as a target gene of the gene expression profiling in the diagnosis method according to the invention.

By performing the gene expression profiling process on a gene group correlated with an indicator indicating a nutritive condition such as PEM (Protein Energy Malnutrition), it is possible to embody a diagnosis method based on a new indicator from a viewpoint different from that of the conventional diagnosis markers. By performing a gene expression profiling process on the gene group correlated with the inflammatory cytokine, it is possible to understand the pathological condition of uremia of a terminal patient with renal failure. By performing a gene expression profiling of a specific gene group correlated with infectious diseases such as pneumonia or bronchitis specific to the aged, it is possible to understand the liability thereof.

By performing a gene expression profiling process of one or more genes of which the expression level significantly varies depending on the kinds of the dialysis membrane used, it is possible to use the diagnosis method according to the invention as a method of selecting dialysis membrane.

The expression products of one or more genes used in the diagnosis method according to the invention can be selected based on a general statistical technique. By feeding back a patient's profiling data, which is obtained by performing a gene expression profiling process using the expression products of a selected specific gene, to other clinical data, it is possible to select a marker which can be used for more accurate diagnosis. If necessary, it is possible to select a marker which can provide a result with higher precision by repeating the process of feeding back the result to other clinical data. Similarly, it is also possible to add a marker corresponding to a new clinical indicator or to add a new marker corresponding to the same clinical indicator.

The blood diagnosis method according to the invention is performed by performing a gene analysis using a gene diagnosis tem directly on a blood sample or on the blood sample having been subjected to pretreatment. This method can be embodied by performing the collection of the blood samples in the step of (1) by the use of a tube properly branched from a dialyzer, which the dialysis patient's blood is made to flow in, to the outside thereof. The collected blood sample is processed and detected in the steps of (2) and (3) by the use of an integrated cartridge which has means for extracting the mRNAs from the blood, means for detecting the mRNAs from blood, and individual chambers connected to each other through flow passages so as to implement the means.

An example of the integrated cartridge which can be used in the blood diagnosis method according to the invention is disclosed in Patent Literature 2.

In the blood diagnosis method according to the present invention, the following advantages are obtained by previously selecting the mRNA markers correlated with the clinical data. (1) It is possible to select dialysis membrane corresponding to a patient's condition. Since the diagnosis can be rapidly made by the mRNA marker, it is possible to accurately select the proper dialysis membrane and to optimize the dialysis treatment. (2) It is easy to find out the primary disease of a chronic dialysis patient. (3) It is possible to obtain diagnosis evaluation and dialysis treatment reflecting individual differences. (4) It is possible to prevent complications such as infectious diseases. It is possible to obtain an estimation correlated with the complication, which could not be extracted using the conventional diagnosis marker, or proper dialysis treatment by using the mRNA marker which is an indicator of the complication. (5) It is possible to establish a proper treatment plan by means of identification of a cause. For example, using an mRNA marker corresponding to clinical data, it is possible to determine the causes of chronic nephritis, renal disease derived from diabetics, and the like. (6) It is possible to determine a patient's nutritive condition. As described above, by using the gene group correlated with an indicator indicating the nutritive condition such as PEM as the mRNA marker, it is possible to improve the medical condition with improvement of the nutritive condition. (7) Since the relationship between a patient's primary disease or medical condition and a treatment effect due to the dialysis on the patient is found out by the correlation accumulation of the clinical data and the mRNA profiles, it is possible to properly determine a start time of dialysis. Accordingly, it is possible to continue a remedy of chronic renal failure in pre-dialysis phase for a long time.

In the blood diagnosis method according to the invention, since the blood as a dialysis target is used as a sample, it is possible to more directly and efficiently determine the effects of dialysis in comparison with diagnoses using other clinical data. Since the effect of dialysis is rapidly reflected in the blood sample, it is possible to make a rapid diagnosis.

Since the mRNA is selected from granulocytes (neutrophils) in blood but the generation of mRNA as a target is promoted by a stimulus when the blood passes through dialysis membrane, it is possible to more effectively make a gene analysis.

In the blood diagnosis method according to the present invention, it is possible to determine a patient's inflammatory condition, nutritive condition, and sarcolysis condition by properly selecting the mRNA marker. It is possible to determine the generation condition of inflammatory cytokines and thus to determine refractoriness to erythropoietin, resistance to insulin, and inappropriate secretion of adipocytokine.

The present invention provides a blood examination method in addition to the blood diagnosis method. The blood examination method includes: (A) extracting mRNAs from blood samples collected from a patient before and/or after dialysis; and (B) carrying out a gene expression profiling process on the extracted mRNAs before and/or after the dialysis. Similarly to the blood diagnosis method according to the present invention, the step of (B) may be performed on expression products of one or more predetermined genes by the use of a DNA microarray or a real-time PCR. The expression products of the one or more genes may include at least one selected from a group consisting of: (a) one that an expression level in a primary disease is significantly different from that of a normal person; (b) one that an expression level significantly varies depending on severities of a patient's medical condition; (c) one that an expression level significantly varies before and/or after the dialysis depending on the types of dialysis membrane used in the dialysis; and (d) one that an expression level significantly varies in a prognosis. The steps of (A) and (B) may be performed by the use of an integrated cartridge which has means for extracting the mRNAs from the blood, means for detecting the mRNAs from blood, and individual chambers connected to each other through flow passages so as to implement the means.

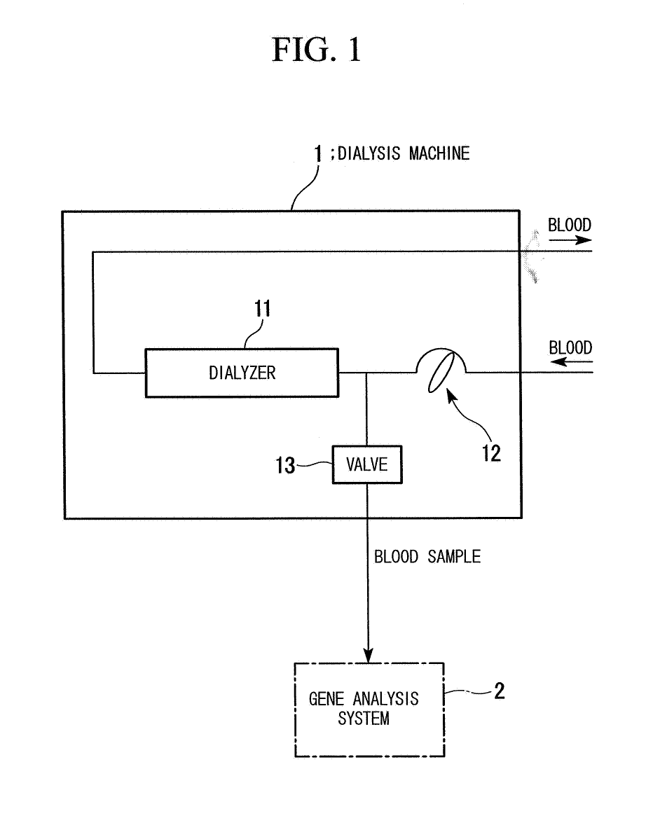

FIG. 1 is a diagram illustrating a configuration of a dialysis machine according to the invention.

The dialysis machine according to the invention includes: an inflow line for allowing blood to flow from a dialysis patient; a dialyzer connected to the inflow line; an outflow line for allowing the blood to flow in the dialysis patient from the dialyzer; and a blood sample collection line branched from the inflow line through a valve. In the dialysis machine, a gene analyzer may be connected to the blood sample collection line. In the dialysis machine, the gene analyzer may include an integrated cartridge which has means for extracting the mRNAs from the blood, means for detecting the mRNAs from blood, and individual chambers connected to each other through flow passages so as to implement the means.

Since the dialysis machine according to the invention has the above-mentioned configuration, a blood sample can be collected directly from the dialysis machine 1. A patient's blood is returned to the patient's body through a blood transmitter 12 and a dialyzer 11 of the dialysis machine 1. As shown in FIG. 1, a valve 13 for collecting a blood sample is disposed in front of the dialyzer 11 and it is thus possible to collect a patient's blood for diagnosis or examination by opening the valve 13 at the time of starting or ending the dialysis. In the invention, “before and/or after dialysis” means not only that a blood sample is collected be starting all the dialysis operations and after ending all the dialysis operation, but also that a blood sample is collected several times during the dialysis operation at intervals of time. That is, the blood sample may be collected during the dialysis. By collecting a blood sample during dialysis and then examining the collected blood sample by the use of a gene analysis system 2, it is possible to monitor the patient's condition during the dialysis.

By using the dialysis machine 1 shown in FIG. 1, it is possible to reduce a patient's burden in collecting a blood sample to the minimum. No labor is required to collect a blood sample. The blood sample collected by the dialysis machine 1 may be automatically introduced into the gene analysis system 2. In this case, it is possible to suppress the necessary amount of blood samples.

The gene analysis system 2 may be formed of an integrated cartridge (for example, see Patent Literature 2). Since the integrated cartridge can automatically perform from an extraction of mRNAs from blood to a detection of the mRNAs, a deviation caused by an operator can be reduced. Since a necessary reagent can be built in the integrated cartridge, it is possible to prevent the contamination of the reagent.

Since viruses may be mixed into a patient's blood sample, the treatment thereof is very dangerous. However, when using the integrated cartridge, the processed sample and waste can be discarded in the unit of cartridge, thereby safely treating the blood sample.

As described above, in the blood diagnosis and examination methods and the dialysis machine according to the present invention, since a diagnosis of a collected blood sample is made on the basis of an mRNA marker, it is possible to obtain a diagnosis result which is general, simple, and useful.

The invention is not limited to the above-mentioned embodiment. The invention can be widely used in blood diagnosis methods of making a diagnosis based on blood collected from a dialysis patient.

What is claimed is:

1. A blood diagnosis method comprising the steps of:
(1) collecting blood samples from a dialysis patient before and/or after dialysis;
(2) extracting mRNAs from the collected blood samples; and
(3) carrying out a gene expression profiling process on the extracted mRNAs before and/or after the dialysis.

2. The blood diagnosis method according to claim 1, wherein the step of (3) is performed on expression products of one or more predetermined genes by the use of a DNA microarray or a real-time PCR.

3. The blood diagnosis method according to claim 2, wherein the expression products of the one or more genes include at least one selected from the group consisting of:
(a) one that an expression level in a primary disease is significantly different from that of a normal person;
(b) one that an expression level significantly varies depending on severities of a patient's medical condition;
(c) one that an expression level significantly varies before and/or after the dialysis depending on the types of dialysis membrane used in the dialysis; and
(d) one that an expression level significantly varies in a prognosis.

4. The blood diagnosis method according to claim 1, wherein the collection of the blood samples in the step of (1) is performed by the use of a tube properly branched from a dialyzer, which the dialysis patient's blood is made to flow in, to the outside thereof.

5. The blood diagnosis method according to claim 1, wherein the steps of (2) and (3) are performed by the use of an integrated cartridge which has means for extracting the mRNAs from the blood means for detecting the mRNAs from blood, and individual chambers connected to each other through flow passages so as to implement the means.

6. A blood examination method comprising the steps of:
(A) extracting mRNAs from blood samples collected from a patient before and/or after dialysis; and
(B) carrying out a gene expression profiling process on the extracted mRNAs before and/or after the dialysis.

7. The blood examination method according to claim 6, wherein the step of (B) is performed on expression products of one or more predetermined genes by the use of a DNA microarray or a real-time PCR.

8. The blood examination method according to claim 7, wherein the expression products of the one or more genes include at least one selected from the group consisting of:
(a) one that an expression level in a primary disease is significantly different from that of a normal person;
(b) one that an expression level significantly varies depending on severities of a patient's medical condition;
(c) one that an expression level significantly varies before and/or after the dialysis depending on the types of dialysis membrane used in the dialysis; and
(d) one that an expression level significantly varies in a prognosis.

9. The blood examination method according to claim 6, wherein the steps of (A) and (B) are performed by the use of an integrated cartridge which has means for extracting the mRNAs from the blood, means for detecting the mRNAs from blood, and individual chambers connected to each other through flow passages so as to implement the means.

10. A dialysis machine comprising:
an inflow line for allowing blood to flow from a dialysis patient;
a dialyzer connected to the inflow line;
an outflow line for allowing the blood to flow in the dialysis patient from the dialyzer; and
a blood sample collection line branched from the inflow line through a valve.

11. The dialysis machine according to claim 10, wherein a gene analyzer is connected to the blood sample collection line.

12. The dialysis machine according to claim 11, wherein the gene analyzer includes an integrated cartridge which has means for extracting the mRNAs from the blood, means for detecting the mRNAs from the blood, and individual chambers connected to each other through flow passages so as to implement the means.
